(19) United States
(12) Patent Application Publication
(10) Pub. No.: US 2025/0308644 A1
(43) Pub. Date: Oct. 2, 2025

(54) Method for Determining a Chemotypic Profile

(71) Applicant: Agriculture Victoria Services Pty Ltd, Bundoora (AU)

(72) Inventors: Aaron Christopher Elkins, Taylors Hill (AU); Simone Jane Rochfort, Reservoir (AU); Noel Cogan, Macleod (AU); German Carlos Spangenberg, Bundoora (AU); Doris Sanjeeta Ram, Craigieburn (AU)

(21) Appl. No.: 19/229,113

(22) Filed: Jun. 5, 2025

Related U.S. Application Data
(63) Continuation of application No. 17/297,738, filed on May 27, 2021, now abandoned.

(30) Foreign Application Priority Data
Dec. 14, 2018 (AU) 2018904756

(51) Int. Cl. G16C 20/70; G01N 21/35; G01N 21/3563; G01N 33/00

(52) U.S. Cl. CPC G16C 20/70; G01N 21/3563; G01N 33/0098; G01N 2021/3595; G01N 2201/0221; G01N 2201/08

(57) ABSTRACT

The present invention relates to determining the chemotype profile of cannabis plant material through determining cannabinoid content of the plant material using near infrared spectroscopy. The invention also involves the training of a classifier to determine the chemotype profile of a cannabis plant from the spectroscopic data.

### CROSS-REFERENCE TO RELATED APPLICATIONS

This application is a continuation of U.S. application Ser. No. 17/297,738 filed May 27, 2021, which is a U.S. National Stage Application of PCT Application No. PCT/AU2019/051345 filed Dec. 9, 2019, which claims priority to Australian Provisional Patent Application No. 2018904756 filed Dec. 14, 2018, the disclosure of which applications are hereby expressly incorporated herein by reference in its entirety.

### FIELD

The present invention relates generally to methods for determining the chemotypic profile of cannabis plant material, including uses thereof.

### BACKGROUND

Cannabis is an herbaceous flowering plant of the Cannabis genus (Rosale) that has been used for its fibre and medicinal properties for thousands of years. The medicinal qualities of cannabis have been recognised since at least 2800 BC, with use of cannabis featuring in ancient Chinese and Indian medical texts. Although use of cannabis for medicinal purposes has been known for centuries, research into the pharmacological properties of the plant has been limited due to its illegal status in most jurisdictions.

The chemistry of cannabis is varied. It is estimated that cannabis plants produce more than 400 different molecules, including phytocannabinoids, terpenes and phenolics. Cannabinoids, such as Δ-9-tetrahydrocannabinol (THC) and cannabidiol (CBD) are the most well-known and researched cannabinoids. CBD and THC are naturally present in their acidic forms, Δ-9-tetrahydrocannabinolic acid (THCA) and cannabidiolic acid (CBDA) in planta which are alternative products of a shared precursor, cannabigerolic acid (CBGA). Cannabis is often divided into categories based on the abundance of THC and CBD, in particular, Type I cannabis is THC-predominant, Type II cannabis contains both THC and CBD, and Type III is CBD-predominant.

While both the acid and corresponding neutral species of cannabinoids have been reported to have biological activity, it is the neutral forms that are more commonly associated with the effects of cannabis. The acid forms degrade naturally to the corresponding neutral forms at a slow rate via non-enzymatic processes. Typically, however, the rate of decarboxylation is increased by heating (e.g., when smoked), which liberates the neutral cannabinoid analogues to facilitate the biological activity. For example, THCA decarboxylates to its neutral form, THC, which is responsible for the psychoactive properties of cannabis. For some medicinal cannabis preparations (e.g., oil and resin preparations that are not heated for consumption), it is necessary that the cannabis material from which these preparations are derived are ‘cured’ (i.e., heated under controlled conditions) to ensure maximum decarboxylation of cannabinoids prior to consumption.

Many cannabinoids interact with the endocannabinoid system in mammals, including humans, to exert complex biological effects on the neuronal, metabolic, immune and reproductive systems. They also interact with G protein-coupled receptors (GPCRs), such as CB1 and CB2, in the human endocannabinoid system, where they are thought to play a part in the regulation of appetite, pain, mood, memory, inflammation and insulin sensitivity. Cannabinoids have also been implicated in neuronal signalling, gastrointestinal inflammation, tumorigenesis, microbial infection and diabetes.

Since different cannabinoids are likely to have different therapeutic potential, it is important to be able to screen and select for cannabis strains that have the desirable chemotypic (cannabinoid) profiles that make them suitable for medicinal use. Previous studies of the cannabinoid content of cannabis plants have largely focused on the differentiation of cannabis varieties bred for recreational or industrial use. For example, in a study conducted by Turner et al. (1979, Journal of Natural Products, 42:319-21), leaf material from 85 cannabis varieties was screened for cannabichromene (CBC), CBD and THC in order to differentiate between recreational and industrial cannabis varieties. The recreational varieties were subjected to further cannabinoid testing to identify the correct time for sampling due to the significant variation of cannabinoid biosynthesis over the life of the plant. In this context, time of sampling is important since the levels of cannabinoids vary significantly. Furthermore, any early reports of looking at CBD levels are likely to be inaccurate since CBC had been previously been misidentified as CBD. More recently, nuclear magnetic resonance (NMR) spectroscopy and RT-PCR analysis has been used to investigate the metabolome and cannabinoid biosynthesis in the trichomes of Cannabis sativa “Bebiol” in the last four weeks (i.e., week five to week nine) of the flowering period (Happyana and Kayser, 2016, Planta Medica, 82:1217-23). In this study, cannabinoid biosynthesis increased in week five to six but was relatively static in the later weeks once the buds were mature. Following biosynthesis, there is a slow decline in certain cannabinoids, particularly THC as the plant material ages.

There remains, therefore, an urgent need for improved tools and methods for measuring cannabinoids in plant material, and in a manner that is suitable for use in production systems (e.g., glasshouses, greenhouses) to assist producers to optimise the value of their crop.

### SUMMARY

In an aspect disclosed herein, there is provided a method for determining a chemotypic profile of cannabis plant material, the method comprising:
(a) providing a predetermined association between spectroscopic data from reference cannabis plant material and a chemotypic profile from the reference cannabis plant material, wherein the chemotypic profile evaluates at least one cannabinoid in acid form;
(b) obtaining spectroscopic data from at least one region of sample plant material; and
(c) utilising the predetermined association to determine the chemotypic profile of the sample plant material based on the sample spectroscopic data.

In another aspect disclosed herein, there is provided a method for monitoring a cannabis plant for a change in the chemotypic profile of the cannabis plant, the method comprising:
(a) determining the chemotypic profile of plant material derived from a cannabis plant in accordance with the methods disclosed herein; and
(b) determining the chemotypic profile of plant material derived from the same cannabis plant as (a) in accordance with the methods disclosed herein and at a subsequent time point in the growth cycle of the plant;
(c) comparing the chemotypic profiles determined at (a) and (b) to evaluate whether there has been a change to the chemotypic profile of the cannabis plant.

In another aspect disclosed herein, there is provided a method of selecting growing conditions that favour the development of a cannabis plant with a desirable chemotypic profile, the method comprising:
(a) exposing a first cannabis plant to a first set of selected growing conditions for a period of time;
(b) exposing a second cannabis plant to a second set of growing conditions for a period of time, wherein the second set of selected growing conditions is different from the first set of growing conditions;
(c) optionally, repeating step (b) for a subsequent set of growing conditions that is different from the first and second sets of selected growing conditions;
(d) determining the chemotypic profiles of plant material derived from the cannabis plants exposed to the set of selected growing conditions of steps (a)-(c) in accordance with the methods disclosed herein; and
(e) selecting from the set of growing conditions of steps (a)-(c) one or more sets of selected growing conditions that favor the development of a cannabis plant with a desirable chemotypic profile based on the chemotypic profiles determined at step (d).

In another aspect disclosed herein, there is provided a method of training a classifier to determine the chemotypic profile of cannabis plant material, the method comprising:
(a) obtaining spectroscopic data from cannabis plant material derived from a plurality of cannabis plants and chemotypic profiles from the cannabis plant material, wherein the chemotypic profiles evaluate at least one cannabinoid in acid form;
(b) for each of the plurality of cannabis plant, using a processor, generating an association between the spectroscopic data and the chemotypic profile;
(c) using the association generated in step (b) to train the classifier to determine the chemotypic profile of sample cannabis plant material from spectroscopic data; and
(d) optionally, repeating steps (a)-(c) using a different plurality of cannabis plants to improve the accuracy of the classifier.

The present disclosure also extends to trained classifiers produced from the method of training a classifier, as described herein.

In another aspect disclosed herein, there is provided a method for determining a chemotypic profile of cannabis plant material, the method comprising:
(a) obtaining spectroscopic data from at least one region of the cannabis plant material;
(b) utilising the trained classifier disclosed herein to determine the chemotypic profile of the cannabis plant material from the spectroscopic data, wherein the chemotypic profile comprises at least one cannabinoid in acid form; and
(c) outputting the chemotypic profile.

### BRIEF DESCRIPTION OF THE DRAWINGS

FIG. 1 shows the correlation between the concentration of cannabinoids as measured using LC-MS and the spectra measured using NIR for five different cannabis strains.

FIG. 2 shows the predicted strain type (I, II, III) for five different cannabis strains as determined from NIR spectra following Partial Least Squares Discriminant Analysis (PLS-DA) and venetian blind validation using associations between the cannabinoid concentration measured by LC-MS and the spectra measured by NIR, cross validated (CV) predictions (y-axis) against sample number (x-axis) are shown. The R² for each CV class prediction are 0.96 (I), 1.00 (II) and 0.94 (III), respectively.

FIG. 3 shows the predicted cannabinoid concentration of CBDA using Partial Least Squares (PLS) regression analysis with leave out cross-validation using NIR spectra (y-axis) and CBDA concentration measured by LC-MS (x-axis). The R² for calibration is 1.00 and 0.99 for cross validated predictions.

FIG. 4 shows the correlation between the concentration of cannabinoids as measured using LC-MS and the spectra measured using NIR for 65 different cannabis strains.

FIG. 5 shows the predicted strain type (I, II) for 19 different cannabis strains (x-axis) from the spectra measured by NIR. Strain type was predicted using PLS-DA and venetian blind validation (y-axis). The data shown does not include the strains used in the calibration set. All strain types were correctly predicted (i.e., an error of classification after cross validation of 0%).

FIG. 6 shows the accuracy of the predicted cannabinoid concentration for THCA-A and CBDA using PLS regression analysis using NIR spectra (y-axis) and the THCA-A and CBDA concentration measured by LC-MS (x-axis) for different cannabis strains in the calibration set. The R² for prediction is 0.98 for both THCA-A and CBDA for cross validated predictions.

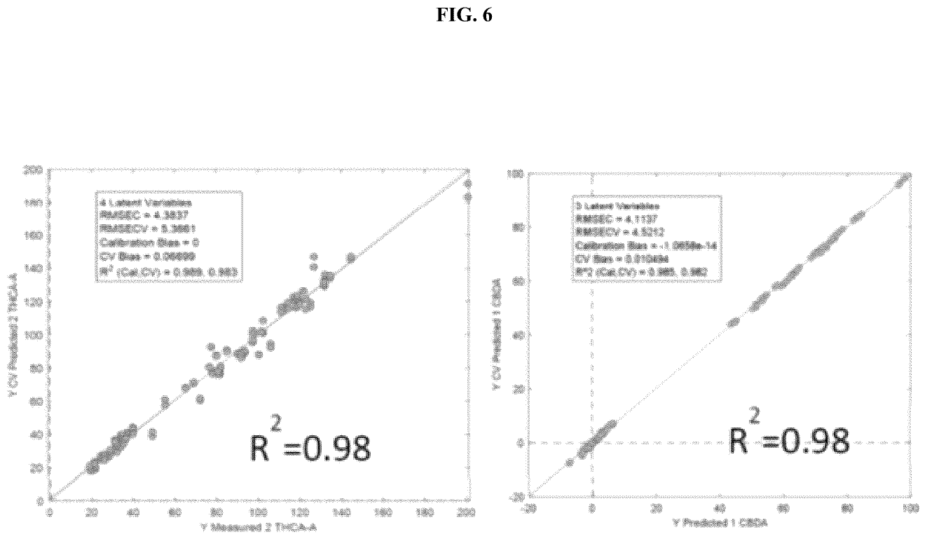

FIG. 7 shows the accuracy of the predicted cannabinoid concentration for THCA-A and CBDA using PLS regression analysis using NIR spectra (y-axis) and the THCA-A and CBDA concentration measured by LC-MS (x-axis) for different cannabis strains in the prediction set (i.e., cannabis strains that are naïve to the model). The R² for prediction is 0.95 for THCA-A and 0.92 for CBDA for cross validated predictions.

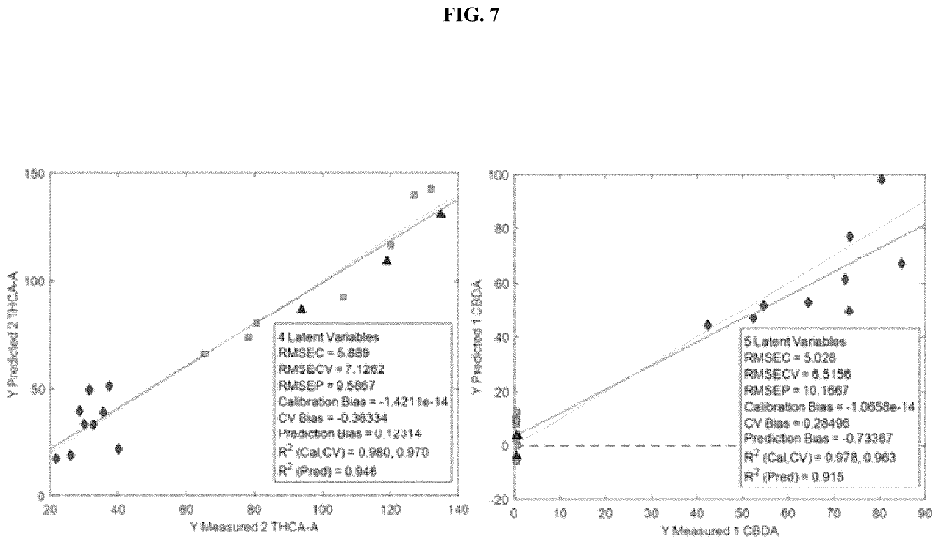

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Unless specifically defined otherwise, all technical and scientific terms used herein shall be taken to have the same meaning as commonly understood by one of ordinary skill in the art.

Unless otherwise indicated the molecular biology, cell culture, laboratory, plant breeding and selection techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T.A. Brown (editor), Essential Molecular Biology: A Practical Approach, Volumes 1 and 2, IRL Press (1991), D.M. Glover and B.D. Hames (editors), DNA Cloning: A Practical Approach, Volumes 1-4, IRL Press (1995 and 1996), and F.M. Ausubel et al. (editors), Current Protocols in Molecular Biology, Greene Pub. Associates and Wiley-Interscience (1988, including all updates until present); Janick, J. (2001) Plant Breeding Reviews, John Wiley & Sons, 252 p.; Jensen, N.F. ed. (1988) Plant Breeding Methodology, John Wiley & Sons, 676 p., Richard, A.J. ed. (1990) Plant Breeding Systems, Unwin Hyman, 529 p.; Walter, F.R. ed. (1987) Plant Breeding, Vol. I, Theory and Techniques, MacMillan Pub. Co.; Slavko, B. ed. (1990) Principles and Methods of Plant Breeding, Elsevier, 386 p.; and Allard, R.W. ed. (1999) Principles of Plant Breeding, John-Wiley & Sons, 240 p. The ICAC Recorder, Vol. XV no. 2:3-14; all of which are incorporated by reference. The procedures described are believed to be well known in the art and are provided for the convenience of the reader. All other publications mentioned in this specification are also incorporated by reference in their entirety.

As used in the subject specification, the singular forms “a”, “an” and “the” include plural aspects unless the context clearly dictates otherwise. Thus, for example, reference to “a plant” includes a single plant, as well as two or more plants; reference to “an inflorescence” includes a single inflorescence, as well as two or more inflorescences; and so forth.

The present disclosure is predicated, at least in part, on the unexpected finding that the chemotypic profile of cannabis plant material that evaluates at least one cannabinoid in acid form can be predicted from spectroscopic data obtained from that plant material by using a predetermined association (e.g., a trained classifier) between spectroscopic data and corresponding chemotypic data of reference cannabis plant material.

Therefore, in an aspect disclosed herein, there is provided a method for determining a chemotypic profile of cannabis plant material, the method comprising:
(a) providing a predetermined association between spectroscopic data from reference cannabis plant material and a chemotypic profile from the reference cannabis plant material, wherein the chemotypic profile evaluates at least one cannabinoid in acid form;
(b) obtaining spectroscopic data from at least one region of sample plant material; and
(c) utilising the predetermined association to determine the chemotypic profile of the sample plant material based on the sample spectroscopic data.

### Cannabis

As used herein, the term “cannabis plant” means a plant of the genus Cannabis, illustrative examples of which include Cannabis sativa, Cannabis indica and Cannabis ruderalis. Cannabis is an erect annual herb with a dioecious breeding system, although monoecious plants exist. Wild and cultivated forms of cannabis are morphologically variable, which has resulted in difficulty defining the taxonomic organisation of the genus. In an embodiment, the cannabis plant is C. sativa.

The terms “plant”, “cultivar”, “variety”, “strain” or “race” are used interchangeably herein to refer to a plant or a group of similar plants according to their structural features and performance (i.e., morphological and physiological characteristics).

The reference genome for C. sativa is the assembled draft genome and transcriptome of “Purple Kush” or “PK” (van Bakal et al. 2011, Genome Biology, 12: R102). C. sativa, has a diploid genome (2n=20) with a karyotype comprising nine autosomes and a pair of sex chromosomes (X and Y). Female plants are homogametic (XX) and males heterogametic (XY) with sex determination controlled by an X-to-autosome balance system. The estimated size of the haploid genome is 818 Mb for female plants and 843 Mb for male plants.

As used herein, the term “plant part” refers to any part of the plant, illustrative examples of which include an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. The term “plant part” also includes any material listed in the Plant Part Code Table as approved by the Australian Therapeutic Goods Administration (TGA) Business Services (TBS). In an embodiment, the part is selected from the group consisting of an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. In a preferred embodiment, the part is a cannabis bud.

### Cannabinoids

The term “cannabinoid”, as used herein, refers to a family of terpeno-phenolic compounds, of which more than 100 compounds are known to exist in nature. Cannabinoids will be known to persons skilled in the art, illustrative examples of which are provided in Table 1, below, including acidic and decarboxylated (i.e., neutral) forms thereof.

TABLE 1
Cannabinoids and their properties.

| | | Chemical |
|---|---|---|
| | | properties/ |
| | | [M + H]⁺ ESI |
| Name | Structure | MS |
| Δ9-tetrahydrocannabinol (THC) | 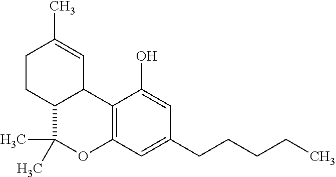 | Psychoactive, decarboxylation product of THCA m/z 315.2319 |
| Δ9- tetrahydrocannabinolic acid (THCA/THCA-A) | 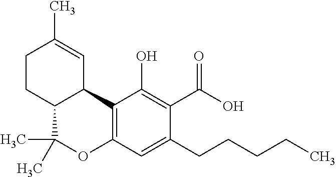 [SMILES: CCCCCc1cc2c(c(O)c1C(=O)O)[C@@H]1C=C(C)CC[C@H]1C(C)(C)O2] | m/z 359.2217 |
| cannabidiol (CBD) | 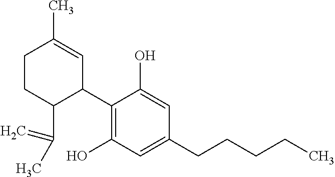 [SMILES: C=C(C)C1CCC(C)=CC1c1c(O)cc(CCCCC)cc1O] | decarboxylation product of CBDA m/z 315.2319 |
| cannabidiolic acid (CBDA) | 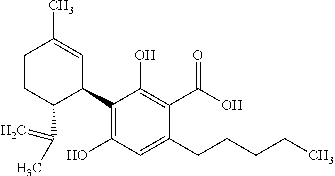 [SMILES: C=C(C)[C@@H]1CCC(C)=C[C@H]1c1c(O)cc(CCCCC)c(C(=O)O)c1O] | m/z 359.2217 |
| cannabigerol (CBG) | 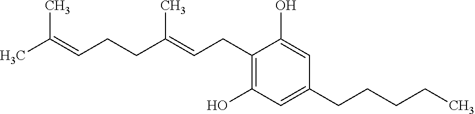 [SMILES: CCCCCc1cc(O)c(C/C=C(\C)CCC=C(C)C)c(O)c1] | Non- intoxicating, decarboxylation product of CBGA m/z 317.2475 |
| cannabigerolic acid (CBGA) | 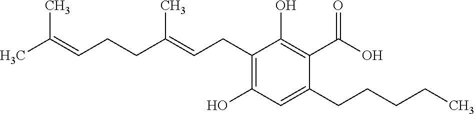 [SMILES: CCCCCc1cc(O)c(C/C=C(\C)CCC=C(C)C)c(O)c1C(=O)O] | m/z 361.2373 |
| cannabichromene (CBC) | 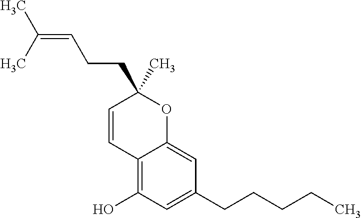 [SMILES: CCCCCc1cc(O)c2c(c1)O[C@](C)(CCC=C(C)C)C=C2] | Non- psychotropic, converts to cannabicyclol upon light exposure m/z 315.2319 |
| cannabichromene acid (CBCA) | 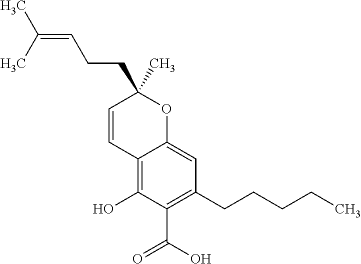 [SMILES: CCCCCc1cc2c(c(O)c1C(=O)O)C=C[C@@](C)(CCC=C(C)C)O2] | m/z 359.2217 |
| cannabicyclol (CBL) | 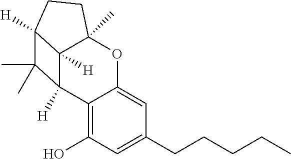 | Non- psychoactive, 16 isomers known. Derived from non- enzymatic conversion of CBC m/z 315.2319 |
| cannabinol (CBN) | 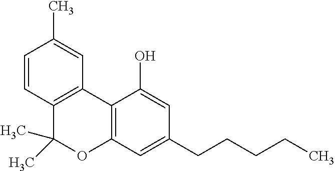 [SMILES: CCCCCc1cc(O)c2c(c1)OC(C)(C)c1ccc(C)cc1-2] | Likely degradation product of THC m/z 311.2006 |
| cannabinolic acid (CBNA) | 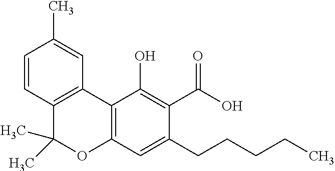 [SMILES: CCCCCc1cc2c(c(O)c1C(=O)O)-c1cc(C)ccc1C(C)(C)O2] | m/z 355.1904 |
| tetrahydrocannabivarin (THCV) | 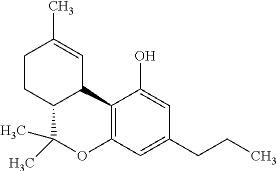 | decarboxylation product of THCVA m/z 287.2006 |
| tetrahydrocannabivarinic acid (THCVA) | 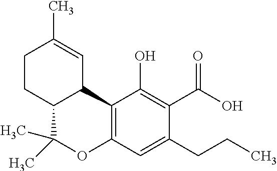 | m/z 331.1904 |
| cannabidivarin (CBDV) | 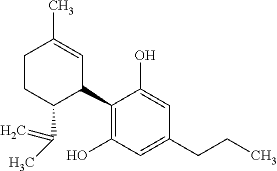 | m/z 287.2006 |
| cannabidivarinic acid (CBDVA) | 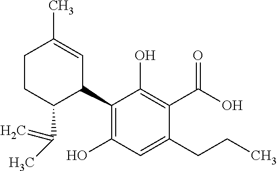 | m/z 331.1904 |
| Δ8-tetrahydrocannabinol (d8-THC) | 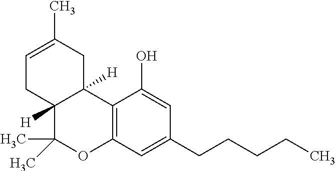 [SMILES: CCCCCc1cc(O)c2c(c1)OC(C)(C)[C@@H]1CC=C(C)C[C@@H]21] | m/z 315.2319 |

Cannabinoids are synthesised in cannabis plants as carboxylic acids. While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying, heating and/or curing (i.e., heating for a specific time and temperature to ensure maximum decarboxylation) the plant material. Persons skilled in the art would be familiar with methods by which decarboxylation of cannabinoids can be promoted, illustrative examples of which include combustion, vaporisation, curing, drying, heating and baking.

“Δ-9-tetrahydrocannabinolic acid” or “THCA-A” is synthesised from the CBGA precursor by THCA synthase. The neutral form “Δ-9-tetrahydrocannabinol” or “THC” is associated with psychoactive effects of cannabis, which are primarily mediated by its activation of CB1G-protein coupled receptors, which result in a decrease in the concentration of cyclic AMP (cAMP) through the inhibition of adenylate cyclase. THC also exhibits partial agonist activity at the cannabinoid receptors CB1 and CB2. CB1 is mainly associated with the central nervous system, while CB2 is expressed predominantly in the cells of the immune system. As a result, THC is also associated with pain relief, relaxation, fatigue, appetite stimulation, and alteration of the visual, auditory and olfactory senses. Furthermore, more recent studies have indicated that THC mediates an anti-cholinesterase action, which may suggest its use for the treatment of Alzheimer's disease and myasthenia (Eubanks et al., 2006, Molecular Pharmaceuticals, 3(6): 773-7).

Acid forms of cannabinoids will be known to persons skilled in the art, illustrative examples of which are described in Papaset et al. (Int. J. Med. Sci., 2018; 15(12): 1286-1295) and Cannabis and Cannabinoids (PDQ®): Health Professional Version; PDQ Integrative, Alternative, and Complementary Therapies Editorial Board; Bethesda (MD): National Cancer Institute (US); 2002-2018).

“Cannabidiolic acid” or “CBDA” is also a derivative of cannabigerolic acid (CBGA), which is converted to CBDA by CBDA synthase. Its neutral form, “cannabidiol” or “CBD” has antagonist activity on agonists of the CB1 and CB2 receptors. CBD has also been shown to act as an antagonist of the putative cannabinoid receptor, GPR55. CBD is commonly associated with therapeutic or medicinal effects of cannabis and has been suggested for use as a sedative, anti-inflammatory, anti-anxiety, anti-nausea, atypical anti-psychotic, and as a cancer treatment. CBD can also increase alertness, and attenuate the memory impairing effect of THC.

In an embodiment, the chemotypic profile evaluates at least one cannabinoid in acid form selected from the group consisting of CBDA, THCA-A, CBDVA, CBGA, THCVA, CBNA and CBCA. In another embodiment, the chemotypic profile evaluates at least one cannabinoid in acid form selected from the group consisting of THCA-A, CBDA, CBGA, CBCA and CBNA. In a preferred embodiment, the chemotypic profile evaluates at least one cannabinoid in acid form selected from the group consisting of THCA-A and CBDA.

In an embodiment, the chemotypic profile evaluates at least one cannabinoid in acid form and at least one cannabinoid in neutral form.

In an embodiment, the at least one cannabinoid in acid form is selected from the group consisting of CBDA, THCA-A, CBDVA, CBGA, THCVA, CBNA and CBCA, and wherein the at least one cannabinoid in neutral form is selected from the group consisting of CBD, THC, CBG, CBDV and THCV. In another embodiment, the at least one cannabinoid in acid form is selected from the group consisting of CBDA, THCA-A, CBGA, CBNA and CBCA, and wherein the at least one cannabinoid in neutral form is selected from the group consisting of CBD and CBDV. In yet another embodiment, the at least one cannabinoid in acid form is selected from the group consisting of THCA-A and CBDA.

By “at least one” means 1, 2, 3, 4, 5, 6, 7, and so on. In an embodiment, the chemotypic profile evaluates at least two, preferably at least three, preferably at least four, preferably at least five, preferably at least six, preferably at least seven, preferably at least eight, preferably at least nine, preferably at least ten, preferably at least eleven cannabinoids, preferably at least twelve, preferably at least thirteen, and more preferably fourteen cannabinoids selected from the group consisting of CBD, CBDA, THC, THCA-A, CBC, CBDVA, CBDV, CBGA, CBG, THCV, THCVA, CBNA, CBN and CBCA. In an embodiment, the chemotypic profile evaluates CBD, CBDA, THC, THCA-A, CBC, CBDV, CBDVA, CBGA, CBG, THCV, THCVA, CBNA and CBCA.

### Chemotypic Profile

The terms “chemotypic profile” or “chemotype” are used interchangeably herein to refer to a representation of the type, amount, level, ratio and/or proportion of cannabinoids that are present in the cannabis plant or part thereof, as typically measured within plant material derived from the plant or plant part, including an extract therefrom.

The chemotypic profile in a cannabis plant will typically predominantly comprise the acidic form of the cannabinoids, but may also comprise some decarboxylated (i.e., neutral) forms thereof, at various concentrations or levels at any given time (i.e., at propagation, growth, harvest, drying, curing, etc).

In an embodiment, the chemotypic profile evaluates the concentration of at least one cannabinoid in the plant material.

The terms “level”, “content”, “concentration” and the like, are used interchangeably herein to describe an amount of the referenced compound, and may be represented in absolute terms (e.g., mg/g, mg/ml, etc) or in relative terms, such as a ratio to any or all of the other compounds in the cannabis plant material or as a percentage of the amount (e.g., by weight) of any or all of the other compounds in the cannabis plant material.

As used herein, the term “plant material” is to be understood to mean any part of the cannabis plant, including the leaves, stems, roots, and inflorescence, or parts thereof, as described elsewhere herein, as well as extracts, illustrative examples of which include kief or hash, which includes trichomes and glands. In an embodiment, the plant material is derived from a female cannabis plant. In another embodiment, the plant material is an inflorescence or a leaf. In a preferred embodiment, the plant material is an inflorescence.

The term “inflorescence” as used herein means the complete flower head of the cannabis plant, comprising stems, stalks, bracts, flowers and trichomes (i.e., glandular, sessile and stalked trichomes). In a preferred embodiment, the plant material comprises cannabis trichomes.

As noted elsewhere herein, cannabinoids are synthesised in cannabis plants predominantly in acid form (i.e., as carboxylic acids). While some decarboxylation may occur in the plant, decarboxylation typically occurs post-harvest and is increased by exposing the plant material to heat. Thus, in an embodiment, the methods disclosed herein comprise obtaining spectroscopic data from plant material that has not been heat treated under conditions and for a period of time that would otherwise result in the decarboxylation of acid forms of cannabinoids in the plant material.

In an embodiment, the chemotypic profile evaluates at least one cannabinoid in acid form and at least one cannabinoid in neutral form.

In an embodiment, the chemotypic profile evaluates at least one cannabinoid in neutral form, preferably at least two, preferably at least three, preferably at least four, preferably at least five, preferably at least six or more preferably at least seven cannabinoids in neutral form.

In an embodiment, the chemotypic profile evaluates at least one, preferably at least two, preferably at least three, preferably at least four, preferably at least five, preferably at least six or more preferably at least seven cannabinoids in neutral form selected from the group consisting of CBD, THC, CBC, CBG, CBDV, THCV and CBN.

In an embodiment, the chemotypic profile evaluates at least one cannabinoid in acid form, preferably at least two, preferably at least three, preferably at least four, preferably at least five, preferably at least six or more preferably at least seven cannabinoids in acid form.

In an embodiment, the chemotypic profile evaluates at least one, preferably at least two, preferably at least three, preferably at least four, preferably at least five, preferably at least six or more preferably at least seven cannabinoids in acid form selected from the group consisting of CBDA, THCA-A, CBDVA, CBGA, THCVA, CBNA and CBCA.

As described elsewhere herein, the chemotypic profile evaluates at least one cannabinoid selected from the group consisting of CBD, CBDA, THC, THCA-A, CBC, CBDV, CBDVA, CBGA, CBG, THCV, THCVA, CBNA and CBCA. In another embodiment, the chemotypic profile evaluates at least one cannabinoid selected from the group consisting of THCA-A, CBDA, CBGA, CBCA, CBNA, CBD and CBDV. In a preferred embodiment, the chemotypic profile evaluates at least one cannabinoid selected from the group consisting of THCA-A and CBDA.

In an embodiment, the chemotypic profile may be used to classify the plant material into Type I (THC/THCA-enriched), Type II (THC/THCA-and CBD/CBDA-enriched) and/or Type III (CBD/CBDA-enriched) cannabis plant material. By “enriched” means that the referenced cannabinoid(s) is/are the main cannabinoid(s) in the plant material.

Methods for measuring a chemotypic profile of a plant or plant part would be familiar to persons skilled in the art, illustrative examples of which include nuclear magnetic resonance (NMR) spectroscopy, RT-PCR analysis, gas chromatography-mass spectroscopy (GC-MS) and liquid chromatography-mass spectroscopy (LC-MS). Other illustrative examples of methods suitable for measuring a chemotypic profile of a cannabis plant, or of a plant part, are described in U.S. Pat. No. 20,150,359188A1, the content of which is incorporated herein by reference.

In an embodiment, the chemotypic profile is measured by LC-MS.

### Infrared Spectroscopy and Near-Infrared Spectroscopy

The present disclosure provides methods for determining a chemotypic profile of cannabis plant material from spectroscopic data. Methods for measuring spectroscopic data would be known to persons skilled in the art, illustrative examples of which include infrared (IR) spectroscopy and near-infrared (NIR) spectroscopy. The principles of IR spectroscopy related to the examination of absorption and transmission of photons in the infrared energy range, based on their frequency and intensity. Different IR-spectra are measured depending on the type of IR used. For example, far-infrared ranges from a frequency of 300 GHz and/or a wavelength of 1 mm to a frequency of 30 THz and/or 10 μm wavelength, mid-infrared ranges from frequencies of 30 to 120 THz and/or wavelengths of 10 to 2.7 μm, and NIR ranges from frequencies of 120 to 400 THz and/or wavelengths of 2,700 to 750 nm.

The term “spectroscopic data” as used herein refers to a spectrum or spectra measured in either reflection or transmission.

In an embodiment, the spectroscopic data is NIR spectrum or spectra. NIR spectra can be used to identify single chemical characteristics of a certain chemical group (i.e., cannabinoids) and more complex characteristics, such as the chemical, structural, sensoric or functional qualities of different cannabis plants.

In an embodiment, the spectroscopic data is measured by near NIR spectroscopy. In another embodiment, the spectroscopic data is measured by Fourier-transform NIR (FT-NIR) spectroscopy, as described, for example, by Maresca M. (2014; Toxins (Basel); 6(11): 3129-3143).

NIR spectroscopy is based on molecular overtone and combination vibrations. Such transitions are forbidden by the selection rules of quantum mechanics. As a result, the molar absorptivity in the NIR region is typically quite small. This is particularly advantageous as NIR can penetrate much further into a sample of cannabis plant material, when compared to mid-infrared radiation, for example. Accordingly, NIR is useful in probing material with little to no sample preparation.

In an embodiment, the spectroscopic data is measured using a rotary cup. In another embodiment, the spectroscopic data is measured using a fibre optic probe.

Apparatus for measuring spectroscopic data would be known to persons skilled in the art, illustrative examples of which include a FT-NIR spectrometer as described elsewhere herein. Instrumentation for NIR spectrometry typically comprises a source, a detector and a dispersive element (e.g., a prism or a diffraction grating) to allow the intensity at different wavelengths to be recorded.

In an embodiment, the spectroscopic data is measured using a hand held device. In a preferred embodiment, the spectroscopic data measured using the hand-held device is processed in a control unit, wherein the control unit is configured to receive and process the measured spectroscopic data to determine the chemotypic profile of the plant material based on the predetermined association between spectroscopic data from reference cannabis plant material and a chemotypic profile from the reference cannabis plant material.

The resolution that the spectroscopic data is measured with will determine the number of data points collected from any given cannabis plant material. In an embodiment, the spectroscopic data is measured with a resolution of 8 cm⁻¹.

The spectroscopic data may be filtered or “pre-processed” prior to determining the chemotypic profile of sample cannabis plant material. In an embodiment, the pre-processing limits the measured spectroscopic data to a spectrum of from about 3500 cm⁻¹ to about 12,500 cm⁻¹. In another embodiment, the pre-processing limits the measured spectroscopic data to a spectrum of from about 4000 cm⁻¹ to about 12,500 cm⁻¹. In a preferred embodiment, the pre-processing limits the measured spectroscopic data to a spectrum of from about 3500 cm⁻¹ to about 9250 cm⁻¹.

In another embodiment, the pre-processing further comprises one or more methods selected from the group consisting of: detrend, extended scatted correction (EMSC), orthogonal signal correction (OSC), 1^{st} or 2^{nd} derivative, smoothing, and mean center.

### Classification and Prediction Methods

In accordance with the methods disclosed herein, a “predetermined association” is utilised to determine the chemotypic profile of sample cannabis plant material. The “predetermined association” is established between spectroscopic data from reference cannabis plant material and a chemotypic profile from the reference cannabis plant material (i.e., the same reference cannabis plant material from which the reference spectroscopic data are derived).

In an embodiment, the predetermined association is a predetermined correlation between spectroscopic data from reference cannabis plant material and a chemotypic profile from the reference cannabis plant material.

In another embodiment, the predetermined association is a trained classifier.

The term “trained classifier” as used herein refers to a classifier that may be used to determine the chemotypic profile of sample cannabis material that has not been subject to a different quantitative method, such as LC-MS. To establish a trained classifier, it is necessary to create a “training set” of reference cannabis plant material to use as a standard. In an embodiment, the classifier is trained using spectroscopic data from a plurality of reference cannabis plant material and chemotypic profiles from the plurality of reference cannabis plant material.

In an embodiment, the classifier is trained using Partial Least Squares Discriminant Analysis (PLS-DA). In another embodiment, the classifier is trained using PLS-DA with venetian blinds cross validation.

In another aspect, there is provided a method of training a classifier to determine the chemotypic profile of cannabis plant material, the method comprising:
(a) obtaining spectroscopic data from cannabis plant material derived from a plurality of cannabis plants and chemotypic profiles from the cannabis plant material, wherein the chemotypic profiles evaluate at least one cannabinoid in acid form;
(b) for each of the plurality of cannabis plant, using a processor, generating an association between the spectroscopic data and the chemotypic profile;
(c) using the association generated in step (b) to train the classifier to determine the chemotypic profile of sample cannabis plant material from spectroscopic data; and
(d) optionally, repeating steps (a)-(c) using a different plurality of cannabis plants to improve the accuracy of the classifier.

In another aspect, there is provided a trained classifier produced according to the methods described herein.

In another aspect, there is provided method for determining a chemotypic profile of cannabis plant material, the method comprising:
(a) obtaining spectroscopic data from at least one region of the cannabis plant material;
(b) utilising the trained classifier disclosed herein to determine the chemotypic profile of the cannabis plant material from the spectroscopic data, wherein the chemotypic profile comprises at least one cannabinoid in acid form; and
(c) outputting the chemotypic profile.

### Methods for Monitoring a Cannabis Plant

The methods disclosed herein may suitably be used to monitor changes to the chemotypic profile of cannabis plants, for example, during their growth cycle. This advantageously allows breeders, cultivators and the like to monitor their crop to ensure their plants retain/maintain the desired chemotype(s) or chemotypic profile(s) and, where necessary, remove and/or discard plants with an undesirable chemotype or chemotypic profile.

Thus, in another aspect disclosed herein, there is provided a method for monitoring a cannabis plant for a change in the chemotypic profile of the cannabis plant, the method comprising:
(a) determining the chemotypic profile of plant material derived from a cannabis plant in accordance with the methods disclosed herein; and
(b) determining the chemotypic profile of plant material derived from the same cannabis plant as (a) in accordance with the methods disclosed herein and at a subsequent time point in the growth cycle of the plant;
(c) comparing the chemotypic profiles determined at (a) and (b) to evaluate whether there has been a change to the chemotypic profile of the cannabis plant.

### Methods of Selecting Growing Conditions

The methods disclosed herein may also suitably be used to select growing conditions (e.g., frequency of watering, water quantity and/or quality; amount and/or type of fertiliser used; etc.) that give rise to or promote the development of cannabis plants with a desired chemotypic profile. This advantageously allows breeders, cultivators and the like to optimise growing conditions to produce cannabis plants with desired chemotype(s) or chemotypic profile(s).

Thus, in another aspect disclosed herein, there is provided a method of selecting growing conditions that favour the development of a cannabis plant with a desirable chemotypic profile, the method comprising: the method comprising:
(a) exposing a first cannabis plant to a first set of selected growing conditions for a period of time;
(b) exposing a second cannabis plant to a second set of growing conditions for a period of time, wherein the second set of selected growing conditions is different from the first set of growing conditions;
(c) optionally, repeating step (b) for a subsequent set of growing conditions that is different from the first and second sets of selected growing conditions;
(d) determining the chemotypic profiles of plant material derived from the cannabis plants exposed to the set of selected growing conditions of steps (a)-(c) in accordance with the methods disclosed herein; and
(e) selecting from the set of growing conditions of steps (a)-(c) one or more sets of selected growing conditions that favour the development of a cannabis plant with a desirable chemotypic profile based on the chemotypic profiles determined at step (d).

The term “selecting” as used herein means the selection of a particular growing condition from one or more different growing conditions based on the chemotypic profile of the cannabis plants that develop following exposure to each growing condition evaluated in accordance with the methods disclosed herein.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

### EXAMPLES

### Materials and Methods

### Reagents and Standards

All HPLC grade reagents, water with 0.1% formic acid (mobile phase A), acetonitrile with 0.1% formic acid (mobile phase B) and methanol were obtained from Fisher Scientific (Fair Lawn, NJ). Primary standards for CBDA and THCA-A in acetonitrile, and CBD, CBN, CBC, THC in methanol, at 1000 μg/mL, were commercially purchased from Novachem Pty Ltd (Heidelberg West, Australia) as distributor for Cerilliant Corporation (Round Rock, Texas). A mixed stock standard at 125 μg/mL CBDA, CBN, CBC, THCA-A and 250 μg/mL CBD, THC in methanol was prepared with working standards at 0.05, 0.125, 0.25, 0.5, 1.25, 2.5 and 50.0 μg/mL for CBDA, CBN, CBC and THCA; and 0.1, 0.25, 0.5, 1.0, 2.5, 5.0 and 100.0 μg/mL for CBD and THC prepared from the mixed stock. Primary standards for THCV, CBDV, CBG, THCVA, CBNA, CBCA, CBGA, CBL and Δ8-THC in methanol, at 1000 μg/mL, were commercially purchased from Novachem Pty Ltd (Heidelberg West, Australia) as distributor for Cerilliant Corporation (Round Rock, Texas). These were combined to make a 100 μg/mL stock (i.e. 100 μL taken and mixed from each). This mixed standard was diluted to 0.1, 0.25, 0.5, 1.0, 2.5, 5.0 and 100.0 μg/mL. All standards were stored at −80° C.

### Sample Preparation

Dried and ground plant material was obtained from the Victorian Government Medicinal Cannabis Cultivation Facility. Mature buds (aged from three to five weeks, depending on the strain) from 65 different cannabis cultivars were analysed. Samples were ground to a fine powder with liquid nitrogen using a SPEX SamplePrep 2010 Geno/Grinder for 1 minute at 1500 rpm. After grinding, 10 mg of each sample was weighed into an Axygen 2.0 mL microcentrifuge tube on a Sartorius BP210D analytical balance. Each sample was extracted with 1 mL of methanol, vortexed for 30 seconds, sonicated for 5 minutes and centrifuged at 13,000 rpm for 5 minutes. The supernatant was transferred to a 2 mL amber HPLC vial and diluted 1:3 for analysis.

### LCMS Analysis

Samples were analysed using a Thermo Scientific (Waltham, MA) Q Exactive Plus Orbitrap mass spectrometer (MS) coupled with Thermo Scientific Vanquish ultra-high performance liquid chromatography (UHPLC) system equipped with degasser, binary pump, temperature controlled autosampler and column compartment, and photodiode array detector (PDA).

Separation was carried out using a C18 column (Phenomenex Luna Omega, 1.6 μm, 150 mm ×2.1 mm) maintained at 30° C. with water and acetonitrile (both with 0.1% formic acid) as mobile phases and a flow rate of 0.3 mL/min. The separation gradient is described in Table 2.

The MS was set to acquire a full range spectrum (80-1,200 m/z) followed by a data independent MS2 spectrum in positive polarity with resolution set to 35,000. The capillary temperature was set to 320° C. with sheath and auxiliary gas at 28 and 15 units respectively and a spray voltage of 4 kV. PDA data acquisition was set to a data collection rate of 5 Hz between 190 and 680 nm.

TABLE 2
Separation gradient for LCMS analysis.

| Time | % A (Water | % B (Acetonitrile |
|---|---|---|
| (min) | with 0.1% FA) | with 0.1% FA) |
| 0 | 60.0 | 40.0 |
| 2.0 | 60.0 | 40.0 |
| 3.0 | 25.0 | 75.0 |
| 10.0 | 10.0 | 90.0 |
| 11.0 | 0.0 | 100.0 |
| 15.0 | 0.0 | 100.0 |
| 15.1 | 60.0 | 40.0 |
| 20.0 | 60.0 | 40.0 |

### NIR Spectral Acquisition

Fourier transform near infrared (FT-NIR) spectra were recorded on a multipurpose analyser (MPA) FT-NIR spectrometer (Bruker Optics GmbH, Ettlingen, Germany) equipped with an integrated Michelson interferometer and a PbS detector. Spectra were collected in diffuse reflectance mode in the wavenumber range 12,500-4000 cm⁻¹ (800-2500 nm), with a resolution of 8 cm⁻¹, using the macro sample integrating sphere or fibre optic probe measurement channels. Ground cannabis material was transferred to a 50 mm cup and measurement acquired with the sample rotating or to a 20 mm vial where measurement was acquired in static mode. The fibre optic probe was placed into the ground cannabis and measurements taken.

### Data Processing

Chemometric Analysis: Data was exported to a CSV file and opened in MATLAB (R2018a, Mathworks). Data was analysed using the PLSToolBox (Version 8.6.1, Eigenvector Research, Inc., USA). Data analysis was on a reduced spectral range (3810 cm⁻¹ to 9010 cm⁻¹). Unless otherwise specified the spectral pre-processing used was: Detrend, 1st Derivative (order: 2, window: 15 pt, tails: polyinterp), Mean Center.

### Example 1-FT-NIR Using 50 mm Rotating Cup

### A. Calibration

The spectra of a ground cannabis plant material from a calibration set of five cannabis strains (Strain Nos. 1, 2, 3, 4, 5) were recorded in triplicate. The concentration of cannabinoids in these samples was determined by LCMS analysis (Table 3).

TABLE 3
Calibration Set Cannabinoid Concentration (mg/g)

| | Strain 1 | Strain 2 | Strain 3 | Strain 4 | Strain 5 |
|---|---|---|---|---|---|
| CBDA | 32.38 | 61.47 | 83.23 | 0.65 | 0.73 |
| CBD | 0.43 | 0.63 | 0.49 | 0.00 | 0.00 |
| THC | 0.06 | 3.37 | 2.81 | 6.14 | 7.33 |
| THCA-A | 1.14 | 35.36 | 44.40 | 129.12 | 148.10 |
| CBC | 0.09 | 0.13 | 0.10 | 0.11 | 0.14 |
| CBDV | 0.75 | 0.26 | 0.18 | 0.00 | 0.00 |
| CBDVA | 0.72 | 0.65 | 1.31 | 4.11 | 3.91 |
| CBGA | 0.06 | 0.39 | 0.39 | 2.28 | 1.97 |
| CBG | 0.00 | 0.01 | 0.00 | 0.02 | 0.02 |
| THCV | 0.04 | 0.26 | 0.18 | 1.12 | 1.16 |
| THCVA | 0.01 | 0.09 | 0.07 | 0.17 | 0.19 |
| CBNA | 2.07 | 3.75 | 4.87 | 0.00 | 0.00 |

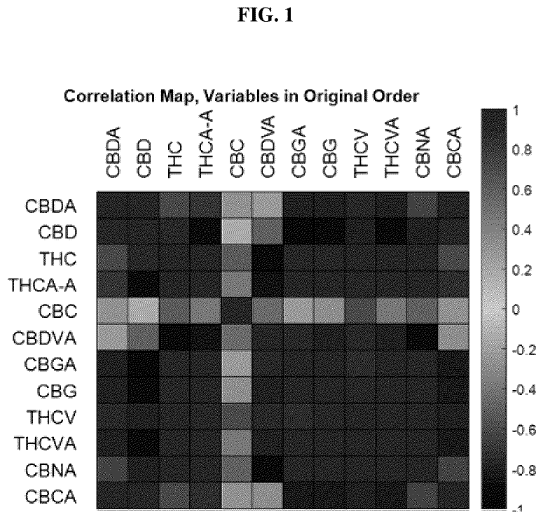

For these strains there was a high correlation between some of the major cannabinoids (THCA-A and CBDA) and the minor cannabinoids (FIG. 1).

This high level of correlation suggests that accurate predictions for the major cannabinoids should be reflected in accurate predictions for cannabinoids present in lower quantities.

### B. Strain Type Identification

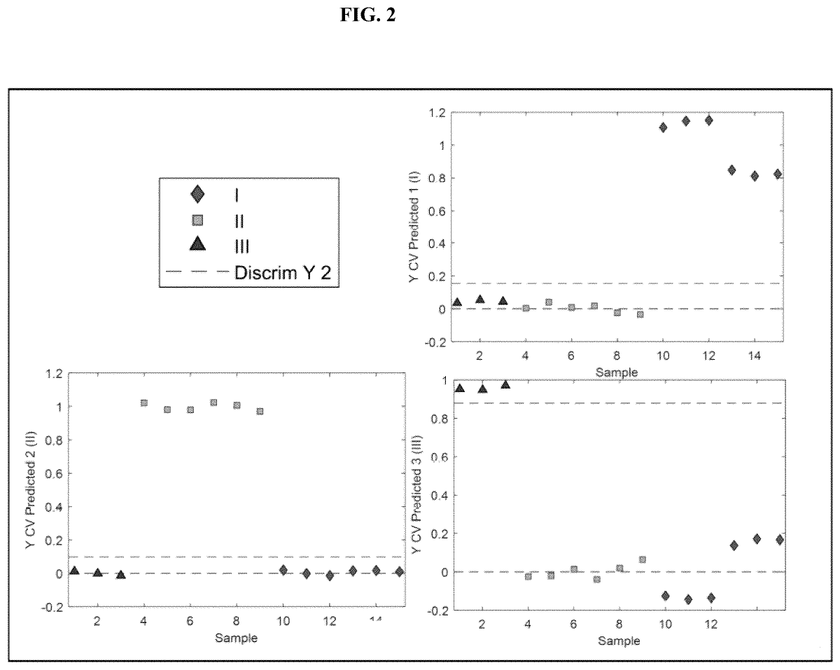

Cannabis is often divided in to categories based on cannabinoid content: Type I cannabis (THC-predominant) and Type II cannabis (containing both THC and CBD) have been described, as well as a Type III, which is rich in CBD. The five strains were classified into each class based on the LCMS data analysis. Strain No. 1 is a Type III strain, Strain Nos. 2 and 3 are Type II strains and Strain Nos. 4 and 5 are Type I strains. Partial Least Squares Discriminant Analysis (PLSDA) with venetian blinds cross validation (7 splits and 1 sample per split) was used to predict each strain type. Classification error was 0%. i.e. each strain type (as defined above) was correctly predicted from the NIR spectra (FIG. 2). Permutation testing (50 iterations) confirmed that the model was not over fitted (p<0.05). This data confirms that NIR can predict strain type.

### C. Cannabinoid Concentration

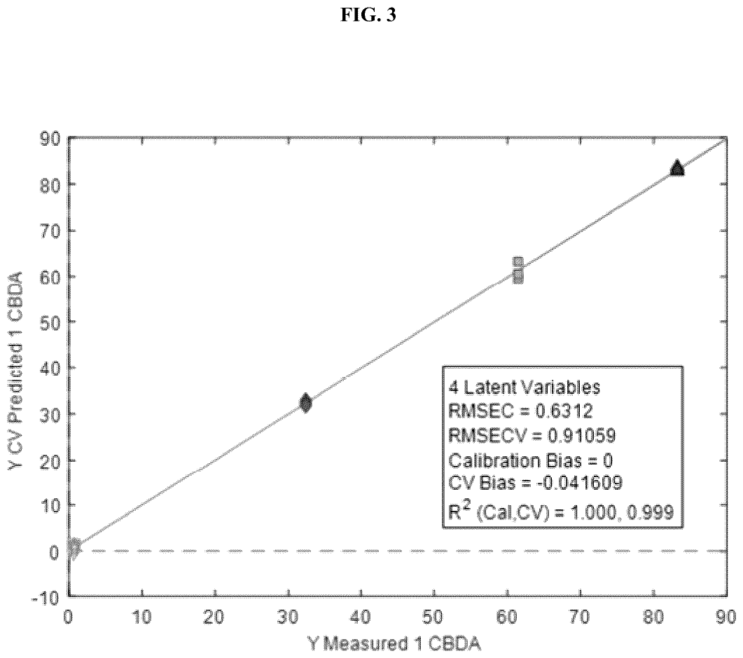

Partial Least Squares (PLS) regression analysis (with leave one out cross validation) of the NIR spectra against the LCMS quantitation data confirmed that cannabinoid concentration can also be predicted from NIR. The CV R² for CBDA, CBD, THC, THCA-A, CBC, CBDVA, CBGA, CBG, THCV, THCVA, CBNA, CBCA measurements were between 0.99 and 1.00. FIG. 3 shows the data for CBDA. Permutation testing (50 iterations) confirmed that the model was not over fitted (p<0.05). This supports the utility of NIR for quantitating the acid form and neutral form of cannabinoids in cannabis plant material.

### Example 2-FT-NIR Using Fibre Optic Probe

### A. Strain Type Identification

As noted in Example 1, above, NIR rotating cup measurements enabled good predictions for strain type and cannabinoid levels. A rotating cup has the advantage of averaging out effects due to sample inhomogeneity. However, using a device such as a fibre optic probe is faster (easier to clean between samples) and more versatile in that the probe can be brought to the sample. The five cannabis strains were therefore tested for type identification and cannabinoid content determination using a fibre optic probe attached to a Bruker MPA FT-NIR spectrometer (Bruker, USA).

Partial Least Squares Discriminant Analysis (PLS-DA) with venetian blinds cross validation (7 splits and 1 sample per split) was used to predict each strain type. Classification error was 0%. i.e., each strain type (as defined above) was correctly predicted from the NIR spectra (FIG. 2). Permutation testing (50 iterations) confirmed that the model was not over fitted (p<0.05). This data confirms the probe provides NIR spectra of sufficient quality to allow strain type prediction.

### B. Cannabinoid Concentration

Partial Least Squares (PLS) regression analysis (with leave one out cross validation) of the probe NIR spectra against the LCMS quantitation data. The CV R² for all the cannabinoids were very good (R²=0.94-0.98) except for CBC which was lower, but still useful (R²=0.79). Permutation testing (50 iterations) confirmed that the model was not over fitted (p<0.05).

### Example 3-Chemotypic Profile of Sample Cannabis Strains

The results of the pilot studies set out in Examples 1 and 2, above, were sufficiently promising that a larger study was undertaken in which 65 different cannabis strains were analysed. The strains are chemotypically diverse (see Table 4) and comprise Type I and Type II strains. Each sample was scanned twice for quality control purposes, but only the first scan was used for model building.

TABLE 4
Prediction Set Cannabinoid Concentration (mg/g)

| Strain # | CBDA | THCA-A | CBD | THC | CBGA | CBG | CBCA | CBC | CBNA | CBN | CBDVA | CBDV | THCVA | THCV |
|---|---|---|---|---|---|---|---|---|---|---|---|---|---|---|
| 2 | 52.31 | 32.58 | 1.03 | 1.38 | 0.79 | 0.42 | 3.01 | 0.11 | 0.09 | 0.02 | 0.22 | 0.01 | 0.22 | 0.01 |
| 2 | 52.31 | 32.58 | 1.03 | 1.38 | 0.79 | 0.42 | 3.01 | 0.11 | 0.09 | 0.02 | 0.22 | 0.01 | 0.22 | 0.01 |
| 3 | 90.54 | 55.48 | 0.88 | 1.72 | 1.87 | 0.65 | 5.27 | 0.12 | 0.04 | 0.01 | 0.20 | 0.00 | 0.32 | 0.00 |
| 3 | 90.54 | 55.48 | 0.88 | 1.72 | 1.87 | 0.65 | 5.27 | 0.12 | 0.04 | 0.01 | 0.20 | 0.00 | 0.32 | 0.00 |
| 6 | 54.58 | 29.90 | 0.91 | 1.46 | 2.00 | 0.38 | 2.96 | 0.12 | 0.08 | 0.01 | 0.28 | 0.01 | 0.33 | 0.02 |
| 6 | 54.58 | 29.90 | 0.91 | 1.46 | 2.00 | 0.38 | 2.96 | 0.12 | 0.08 | 0.01 | 0.28 | 0.01 | 0.33 | 0.02 |
| 7 | 67.83 | 34.29 | 1.43 | 2.40 | 1.83 | 0.53 | 3.82 | 0.19 | 0.09 | 0.02 | 0.32 | 0.02 | 0.26 | 0.02 |
| 7 | 67.83 | 34.29 | 1.43 | 2.40 | 1.83 | 0.53 | 3.82 | 0.19 | 0.09 | 0.02 | 0.32 | 0.02 | 0.26 | 0.02 |
| 8 | 73.31 | 28.52 | 0.83 | 1.24 | 3.20 | 0.44 | 4.28 | 0.12 | 0.13 | 0.01 | 0.36 | 0.01 | 0.32 | 0.01 |
| 8 | 73.31 | 28.52 | 0.83 | 1.24 | 3.20 | 0.44 | 4.28 | 0.12 | 0.13 | 0.01 | 0.36 | 0.01 | 0.32 | 0.01 |
| 9 | 68.63 | 32.06 | 0.88 | 1.31 | 1.88 | 0.67 | 3.66 | 0.11 | 0.12 | 0.01 | 0.31 | 0.01 | 0.34 | 0.01 |
| 9 | 68.63 | 32.06 | 0.88 | 1.31 | 1.88 | 0.67 | 3.66 | 0.11 | 0.12 | 0.01 | 0.31 | 0.01 | 0.34 | 0.01 |
| 10 | 51.43 | 21.93 | 0.54 | 0.75 | 1.80 | 0.31 | 2.64 | 0.07 | 0.08 | 0.01 | 0.29 | 0.00 | 0.25 | 0.01 |
| 10 | 51.43 | 21.93 | 0.54 | 0.75 | 1.80 | 0.31 | 2.64 | 0.07 | 0.08 | 0.01 | 0.29 | 0.00 | 0.25 | 0.01 |
| 11 | 64.89 | 34.26 | 0.82 | 0.82 | 1.98 | 0.25 | 3.36 | 0.10 | 0.08 | 0.01 | 0.31 | 0.01 | 0.28 | 0.01 |
| 11 | 64.89 | 34.26 | 0.82 | 0.82 | 1.98 | 0.25 | 3.36 | 0.10 | 0.08 | 0.01 | 0.31 | 0.01 | 0.28 | 0.01 |
| 12 | 69.76 | 32.28 | 1.11 | 0.86 | 3.38 | 0.34 | 3.85 | 0.14 | 0.09 | 0.01 | 0.35 | 0.02 | 0.34 | 0.01 |
| 12 | 69.76 | 32.28 | 1.11 | 0.86 | 3.38 | 0.34 | 3.85 | 0.14 | 0.09 | 0.01 | 0.35 | 0.02 | 0.34 | 0.01 |
| 13 | 63.49 | 29.20 | 0.78 | 1.06 | 1.61 | 0.29 | 2.96 | 0.08 | 0.12 | 0.01 | 0.34 | 0.01 | 0.31 | 0.01 |
| 13 | 63.49 | 29.20 | 0.78 | 1.06 | 1.61 | 0.29 | 2.96 | 0.08 | 0.12 | 0.01 | 0.34 | 0.01 | 0.31 | 0.01 |
| 14 | 77.35 | 40.01 | 1.02 | 1.58 | 4.10 | 0.38 | 3.81 | 0.13 | 0.13 | 0.02 | 0.39 | 0.02 | 0.40 | 0.02 |
| 14 | 77.35 | 40.01 | 1.02 | 1.58 | 4.10 | 0.38 | 3.81 | 0.13 | 0.13 | 0.02 | 0.39 | 0.02 | 0.40 | 0.02 |
| 15 | 72.47 | 37.31 | 0.59 | 1.02 | 1.68 | 0.68 | 3.70 | 0.07 | 0.07 | 0.01 | 0.39 | 0.00 | 0.44 | 0.01 |
| 15 | 72.47 | 37.31 | 0.59 | 1.02 | 1.68 | 0.68 | 3.70 | 0.07 | 0.07 | 0.01 | 0.39 | 0.00 | 0.44 | 0.01 |
| 16 | 96.15 | 72.30 | 0.82 | 2.18 | 4.33 | 0.80 | 5.11 | 0.12 | 0.12 | 0.01 | 0.46 | 0.01 | 0.48 | 0.02 |
| 16 | 96.15 | 72.30 | 0.82 | 2.18 | 4.33 | 0.80 | 5.11 | 0.12 | 0.12 | 0.01 | 0.46 | 0.01 | 0.48 | 0.02 |
| 17 | 75.78 | 34.91 | 0.94 | 1.51 | 2.19 | 0.68 | 3.93 | 0.12 | 0.08 | 0.01 | 0.36 | 0.01 | 0.30 | 0.01 |
| 17 | 75.78 | 34.91 | 0.94 | 1.51 | 2.19 | 0.68 | 3.93 | 0.12 | 0.08 | 0.01 | 0.36 | 0.01 | 0.30 | 0.01 |
| 18 | 66.77 | 21.83 | 0.80 | 0.47 | 1.59 | 0.55 | 3.59 | 0.11 | 0.07 | 0.01 | 0.39 | 0.01 | 0.40 | 0.01 |
| 18 | 66.77 | 21.83 | 0.80 | 0.47 | 1.59 | 0.55 | 3.59 | 0.11 | 0.07 | 0.01 | 0.39 | 0.01 | 0.40 | 0.01 |
| 19 | 75.54 | 36.30 | 1.07 | 1.62 | 3.15 | 0.49 | 4.60 | 0.15 | 0.08 | 0.01 | 0.38 | 0.02 | 0.33 | 0.02 |
| 19 | 75.54 | 36.30 | 1.07 | 1.62 | 3.15 | 0.49 | 4.60 | 0.15 | 0.08 | 0.01 | 0.38 | 0.02 | 0.33 | 0.02 |
| 20 | 84.84 | 35.58 | 1.40 | 0.83 | 2.42 | 0.71 | 4.88 | 0.19 | 0.09 | 0.02 | 0.42 | 0.02 | 0.51 | 0.02 |
| 20 | 84.84 | 35.58 | 1.40 | 0.83 | 2.42 | 0.71 | 4.88 | 0.19 | 0.09 | 0.02 | 0.42 | 0.02 | 0.51 | 0.02 |
| 21 | 55.46 | 20.02 | 1.26 | 0.84 | 0.98 | 0.08 | 2.92 | 0.15 | 0.06 | 0.02 | 0.10 | 0.00 | 0.27 | 0.00 |
| 21 | 55.46 | 20.02 | 1.26 | 0.84 | 0.98 | 0.08 | 2.92 | 0.15 | 0.06 | 0.02 | 0.10 | 0.00 | 0.27 | 0.00 |
| 22 | 66.93 | 24.62 | 1.22 | 0.76 | 0.99 | 0.18 | 3.96 | 0.15 | 0.09 | 0.02 | 0.12 | 0.00 | 0.29 | 0.00 |
| 22 | 66.93 | 24.62 | 1.22 | 0.76 | 0.99 | 0.18 | 3.96 | 0.15 | 0.09 | 0.02 | 0.12 | 0.00 | 0.29 | 0.00 |
| 23 | 50.26 | 19.45 | 0.93 | 1.04 | 1.79 | 0.12 | 3.04 | 0.12 | 0.07 | 0.01 | 0.09 | 0.00 | 0.16 | 0.00 |
| 23 | 50.26 | 19.45 | 0.93 | 1.04 | 1.79 | 0.12 | 3.04 | 0.12 | 0.07 | 0.01 | 0.09 | 0.00 | 0.16 | 0.00 |
| 24 | 73.48 | 26.01 | 1.26 | 1.34 | 0.91 | 0.36 | 4.08 | 0.16 | 0.09 | 0.02 | 0.14 | 0.00 | 0.27 | 0.00 |
| 24 | 73.48 | 26.01 | 1.26 | 1.34 | 0.91 | 0.36 | 4.08 | 0.16 | 0.09 | 0.02 | 0.14 | 0.00 | 0.27 | 0.00 |
| 25 | 72.85 | 36.80 | 1.07 | 1.75 | 1.56 | 0.39 | 4.13 | 0.14 | 0.10 | 0.01 | 0.13 | 0.00 | 0.25 | 0.00 |
| 25 | 72.85 | 36.80 | 1.07 | 1.75 | 1.56 | 0.39 | 4.13 | 0.14 | 0.10 | 0.01 | 0.13 | 0.00 | 0.25 | 0.00 |
| 26 | 80.42 | 40.04 | 2.04 | 3.30 | 1.12 | 0.34 | 5.90 | 0.31 | 0.08 | 0.03 | 0.19 | 0.01 | 0.25 | 0.02 |
| 26 | 80.42 | 40.04 | 2.04 | 3.30 | 1.12 | 0.34 | 5.90 | 0.31 | 0.08 | 0.03 | 0.19 | 0.01 | 0.25 | 0.02 |
| 27 | 69.26 | 49.44 | 1.16 | 1.33 | 2.38 | 0.39 | 3.72 | 0.32 | 0.05 | 0.02 | 0.38 | 0.02 | 0.30 | 0.03 |
| 27 | 69.26 | 49.44 | 1.16 | 1.33 | 2.38 | 0.39 | 3.72 | 0.32 | 0.05 | 0.02 | 0.38 | 0.02 | 0.30 | 0.03 |
| 28 | 64.37 | 31.51 | 1.04 | 1.63 | 0.68 | 0.26 | 3.30 | 0.11 | 0.17 | 0.02 | 0.30 | 0.02 | 0.34 | 0.01 |
| 28 | 64.37 | 31.51 | 1.04 | 1.63 | 0.68 | 0.26 | 3.30 | 0.11 | 0.17 | 0.02 | 0.30 | 0.02 | 0.34 | 0.01 |
| 29 | 42.34 | 21.72 | 0.77 | 0.67 | 0.94 | 0.23 | 2.45 | 0.10 | 0.09 | 0.01 | 0.20 | 0.00 | 0.20 | 0.01 |
| 29 | 42.34 | 21.72 | 0.77 | 0.67 | 0.94 | 0.23 | 2.45 | 0.10 | 0.09 | 0.01 | 0.20 | 0.00 | 0.20 | 0.01 |
| 30 | 41.93 | 26.37 | 0.89 | 1.99 | 1.00 | 0.30 | 2.11 | 0.12 | 0.10 | 0.02 | 0.21 | 0.01 | 0.26 | 0.02 |
| 30 | 41.93 | 26.37 | 0.89 | 1.99 | 1.00 | 0.30 | 2.11 | 0.12 | 0.10 | 0.02 | 0.21 | 0.01 | 0.26 | 0.02 |
| 31 | 0.55 | 77.63 | 0.00 | 3.12 | 4.41 | 0.41 | 2.85 | 0.09 | 0.13 | 0.03 | 0.00 | 0.00 | 0.26 | 0.01 |
| 31 | 0.55 | 77.63 | 0.00 | 3.12 | 4.41 | 0.41 | 2.85 | 0.09 | 0.13 | 0.03 | 0.00 | 0.00 | 0.26 | 0.01 |
| 32 | 0.51 | 106.13 | 0.00 | 3.98 | 5.19 | 0.97 | 3.39 | 0.12 | 0.17 | 0.03 | 0.00 | 0.00 | 0.23 | 0.01 |
| 32 | 0.51 | 106.13 | 0.00 | 3.98 | 5.19 | 0.97 | 3.39 | 0.12 | 0.17 | 0.03 | 0.00 | 0.00 | 0.23 | 0.01 |
| 34 | 0.68 | 81.89 | 0.00 | 2.33 | 1.93 | 0.39 | 3.38 | 0.07 | 0.24 | 0.02 | 0.00 | 0.00 | 0.16 | 0.00 |
| 34 | 0.68 | 81.89 | 0.00 | 2.33 | 1.93 | 0.39 | 3.38 | 0.07 | 0.24 | 0.02 | 0.00 | 0.00 | 0.16 | 0.00 |
| 36 | 0.27 | 65.34 | 0.00 | 1.10 | 3.17 | 0.82 | 3.29 | 0.09 | 0.15 | 0.01 | 0.00 | 0.00 | 0.29 | 0.01 |
| 36 | 0.27 | 65.34 | 0.00 | 1.10 | 3.17 | 0.82 | 3.29 | 0.09 | 0.15 | 0.01 | 0.00 | 0.00 | 0.29 | 0.01 |
| 37 | 0.99 | 117.13 | 0.00 | 2.77 | 4.54 | 0.85 | 4.70 | 0.15 | 0.12 | 0.02 | 0.00 | 0.00 | 0.64 | 0.02 |
| 37 | 0.99 | 117.13 | 0.00 | 2.77 | 4.54 | 0.85 | 4.70 | 0.15 | 0.12 | 0.02 | 0.00 | 0.00 | 0.64 | 0.02 |
| 38 | 0.70 | 132.30 | 0.00 | 2.24 | 5.30 | 1.50 | 3.17 | 0.11 | 0.19 | 0.02 | 0.00 | 0.00 | 0.73 | 0.02 |
| 38 | 0.70 | 132.30 | 0.00 | 2.24 | 5.30 | 1.50 | 3.17 | 0.11 | 0.19 | 0.02 | 0.00 | 0.00 | 0.73 | 0.02 |
| 39 | 0.41 | 131.97 | 0.00 | 2.27 | 5.45 | 0.78 | 2.60 | 0.07 | 0.18 | 0.01 | 0.00 | 0.00 | 0.92 | 0.02 |
| 39 | 0.41 | 131.97 | 0.00 | 2.27 | 5.45 | 0.78 | 2.60 | 0.07 | 0.18 | 0.01 | 0.00 | 0.00 | 0.92 | 0.02 |
| 40 | 0.46 | 118.36 | 0.00 | 1.39 | 8.60 | 0.38 | 2.80 | 0.10 | 0.22 | 0.01 | 0.00 | 0.00 | 0.89 | 0.02 |
| 40 | 0.46 | 118.36 | 0.00 | 1.39 | 8.60 | 0.38 | 2.80 | 0.10 | 0.22 | 0.01 | 0.00 | 0.00 | 0.89 | 0.02 |
| 41 | 0.38 | 97.60 | 0.00 | 1.56 | 3.91 | 0.62 | 1.55 | 0.05 | 0.17 | 0.01 | 0.00 | 0.00 | 0.49 | 0.01 |
| 41 | 0.38 | 97.60 | 0.00 | 1.56 | 3.91 | 0.62 | 1.55 | 0.05 | 0.17 | 0.01 | 0.00 | 0.00 | 0.49 | 0.01 |
| 42 | 0.55 | 92.31 | 0.00 | 1.06 | 3.86 | 0.49 | 0.86 | 0.03 | 0.20 | 0.01 | 0.00 | 0.00 | 0.61 | 0.01 |
| 42 | 0.55 | 92.31 | 0.00 | 1.06 | 3.86 | 0.49 | 0.86 | 0.03 | 0.20 | 0.01 | 0.00 | 0.00 | 0.61 | 0.01 |
| 43 | 0.38 | 125.19 | 0.00 | 4.09 | 7.92 | 0.36 | 4.59 | 0.30 | 0.15 | 0.03 | 0.00 | 0.00 | 1.36 | 0.04 |
| 43 | 0.38 | 125.19 | 0.00 | 4.09 | 7.92 | 0.36 | 4.59 | 0.30 | 0.15 | 0.03 | 0.00 | 0.00 | 1.36 | 0.04 |
| 44 | 0.34 | 102.53 | 0.00 | 3.18 | 3.25 | 0.28 | 1.53 | 0.10 | 0.13 | 0.03 | 0.00 | 0.00 | 0.76 | 0.02 |
| 44 | 0.34 | 102.53 | 0.00 | 3.18 | 3.25 | 0.28 | 1.53 | 0.10 | 0.13 | 0.03 | 0.00 | 0.00 | 0.76 | 0.02 |
| 45 | 0.25 | 69.22 | 0.00 | 2.31 | 1.55 | 0.68 | 1.59 | 0.07 | 0.19 | 0.02 | 0.00 | 0.00 | 0.27 | 0.01 |
| 45 | 0.25 | 69.22 | 0.00 | 2.31 | 1.55 | 0.68 | 1.59 | 0.07 | 0.19 | 0.02 | 0.00 | 0.00 | 0.27 | 0.01 |
| 46 | 0.36 | 80.71 | 0.00 | 1.02 | 1.27 | 0.41 | 1.03 | 0.06 | 0.17 | 0.02 | 0.00 | 0.00 | 0.36 | 0.01 |
| 46 | 0.36 | 80.71 | 0.00 | 1.02 | 1.27 | 0.41 | 1.03 | 0.06 | 0.17 | 0.02 | 0.00 | 0.00 | 0.36 | 0.01 |
| 47 | 0.39 | 122.93 | 0.00 | 1.47 | 3.05 | 0.44 | 2.88 | 0.08 | 0.20 | 0.03 | 0.00 | 0.00 | 0.69 | 0.02 |
| 47 | 0.39 | 122.93 | 0.00 | 1.47 | 3.05 | 0.44 | 2.88 | 0.08 | 0.20 | 0.03 | 0.00 | 0.00 | 0.69 | 0.02 |
| 48 | 0.41 | 111.63 | 0.00 | 3.41 | 4.14 | 0.74 | 2.15 | 0.11 | 0.15 | 0.02 | 0.00 | 0.00 | 0.67 | 0.02 |
| 48 | 0.41 | 111.63 | 0.00 | 3.41 | 4.14 | 0.74 | 2.15 | 0.11 | 0.15 | 0.02 | 0.00 | 0.00 | 0.67 | 0.02 |
| 49 | 1.05 | 144.60 | 0.00 | 2.34 | 3.58 | 0.68 | 3.49 | 0.10 | 0.23 | 0.03 | 0.00 | 0.00 | 1.16 | 0.03 |
| 49 | 1.05 | 144.60 | 0.00 | 2.34 | 3.58 | 0.68 | 3.49 | 0.10 | 0.23 | 0.03 | 0.00 | 0.00 | 1.16 | 0.03 |
| 51 | 0.42 | 119.98 | 0.00 | 3.05 | 3.81 | 0.57 | 1.45 | 0.08 | 0.29 | 0.03 | 0.00 | 0.00 | 1.10 | 0.03 |
| 51 | 0.42 | 119.98 | 0.00 | 3.05 | 3.81 | 0.57 | 1.45 | 0.08 | 0.29 | 0.03 | 0.00 | 0.00 | 1.10 | 0.03 |
| 52 | 0.62 | 131.87 | 0.00 | 3.09 | 9.05 | 0.64 | 1.52 | 0.06 | 0.18 | 0.03 | 0.00 | 0.00 | 0.83 | 0.02 |
| 52 | 0.62 | 131.87 | 0.00 | 3.09 | 9.05 | 0.64 | 1.52 | 0.06 | 0.18 | 0.03 | 0.00 | 0.00 | 0.83 | 0.02 |
| 53 | 0.35 | 78.28 | 0.00 | 1.47 | 0.81 | 0.99 | 0.99 | 0.08 | 0.14 | 0.04 | 0.00 | 0.00 | 0.48 | 0.03 |
| 53 | 0.35 | 78.28 | 0.00 | 1.47 | 0.81 | 0.99 | 0.99 | 0.08 | 0.14 | 0.04 | 0.00 | 0.00 | 0.48 | 0.03 |
| 54 | 0.54 | 100.53 | 0.00 | 2.99 | 5.26 | 0.75 | 1.71 | 0.10 | 0.08 | 0.01 | 0.00 | 0.00 | 0.50 | 0.02 |
| 54 | 0.54 | 100.53 | 0.00 | 2.99 | 5.26 | 0.75 | 1.71 | 0.10 | 0.08 | 0.01 | 0.00 | 0.00 | 0.50 | 0.02 |
| 55 | 0.46 | 114.45 | 0.00 | 1.59 | 4.51 | 0.77 | 1.64 | 0.09 | 0.11 | 0.03 | 0.00 | 0.00 | 0.73 | 0.02 |
| 55 | 0.46 | 114.45 | 0.00 | 1.59 | 4.51 | 0.77 | 1.64 | 0.09 | 0.11 | 0.03 | 0.00 | 0.00 | 0.73 | 0.02 |
| 56 | 0.49 | 90.21 | 0.00 | 1.54 | 4.00 | 0.56 | 0.91 | 0.06 | 0.10 | 0.02 | 0.00 | 0.00 | 0.45 | 0.02 |
| 56 | 0.49 | 90.21 | 0.00 | 1.54 | 4.00 | 0.56 | 0.91 | 0.06 | 0.10 | 0.02 | 0.00 | 0.00 | 0.45 | 0.02 |
| 57 | 0.49 | 111.21 | 0.00 | 3.18 | 3.57 | 0.90 | 1.29 | 0.09 | 0.12 | 0.02 | 0.00 | 0.00 | 0.74 | 0.02 |
| 57 | 0.49 | 111.21 | 0.00 | 3.18 | 3.57 | 0.90 | 1.29 | 0.09 | 0.12 | 0.02 | 0.00 | 0.00 | 0.74 | 0.02 |
| 58 | 0.50 | 126.99 | 0.00 | 5.06 | 7.07 | 0.66 | 1.56 | 0.12 | 0.07 | 0.04 | 0.00 | 0.00 | 0.64 | 0.03 |
| 58 | 0.50 | 126.99 | 0.00 | 5.06 | 7.07 | 0.66 | 1.56 | 0.12 | 0.07 | 0.04 | 0.00 | 0.00 | 0.64 | 0.03 |
| 59 | 0.77 | 200.89 | 0.00 | 2.69 | 4.21 | 0.60 | 1.83 | 0.15 | 0.16 | 0.05 | 0.00 | 0.00 | 0.91 | 0.03 |
| 59 | 0.77 | 200.89 | 0.00 | 2.69 | 4.21 | 0.60 | 1.83 | 0.15 | 0.16 | 0.05 | 0.00 | 0.00 | 0.91 | 0.03 |
| 60 | 0.73 | 121.89 | 0.00 | 2.20 | 0.89 | 0.94 | 1.72 | 0.05 | 0.20 | 0.01 | 0.02 | 0.00 | 5.38 | 0.08 |
| 60 | 0.73 | 121.89 | 0.00 | 2.20 | 0.89 | 0.94 | 1.72 | 0.05 | 0.20 | 0.01 | 0.02 | 0.00 | 5.38 | 0.08 |
| 61 | 0.68 | 93.82 | 0.00 | 2.49 | 2.45 | 0.29 | 2.22 | 0.07 | 0.28 | 0.03 | 0.02 | 0.00 | 4.43 | 0.11 |
| 61 | 0.68 | 93.82 | 0.00 | 2.49 | 2.45 | 0.29 | 2.22 | 0.07 | 0.28 | 0.03 | 0.02 | 0.00 | 4.43 | 0.11 |
| 62 | 0.46 | 118.93 | 0.00 | 1.94 | 4.96 | 0.34 | 2.07 | 0.05 | 0.23 | 0.02 | 0.02 | 0.00 | 3.20 | 0.07 |
| 62 | 0.46 | 118.93 | 0.00 | 1.94 | 4.96 | 0.34 | 2.07 | 0.05 | 0.23 | 0.02 | 0.02 | 0.00 | 3.20 | 0.07 |
| 63 | 0.80 | 101.70 | 0.00 | 2.88 | 8.06 | 0.83 | 1.84 | 0.10 | 0.16 | 0.03 | 0.03 | 0.00 | 6.39 | 0.18 |
| 63 | 0.80 | 101.70 | 0.00 | 2.88 | 8.06 | 0.83 | 1.84 | 0.10 | 0.16 | 0.03 | 0.03 | 0.00 | 6.39 | 0.18 |
| 64 | 0.42 | 134.75 | 0.00 | 4.41 | 4.04 | 0.43 | 2.15 | 0.17 | 0.16 | 0.03 | 0.02 | 0.00 | 5.46 | 0.17 |
| 64 | 0.42 | 134.75 | 0.00 | 4.41 | 4.04 | 0.43 | 2.15 | 0.17 | 0.16 | 0.03 | 0.02 | 0.00 | 5.46 | 0.17 |
| 65 | 0.29 | 82.13 | 0.00 | 3.54 | 0.99 | 0.48 | 1.12 | 0.09 | 0.09 | 0.03 | 0.02 | 0.00 | 2.62 | 0.12 |
| 65 | 0.29 | 82.13 | 0.00 | 3.54 | 0.99 | 0.48 | 1.12 | 0.09 | 0.09 | 0.03 | 0.02 | 0.00 | 2.62 | 0.12 |
| 66 | 0.66 | 97.54 | 0.00 | 2.18 | 0.88 | 0.49 | 1.26 | 0.06 | 0.10 | 0.02 | 0.02 | 0.00 | 2.71 | 0.08 |
| 66 | 0.66 | 97.54 | 0.00 | 2.18 | 0.88 | 0.49 | 1.26 | 0.06 | 0.10 | 0.02 | 0.02 | 0.00 | 2.71 | 0.08 |
| 67 | 0.61 | 85.13 | 0.00 | 1.56 | 1.03 | 0.46 | 0.94 | 0.05 | 0.12 | 0.02 | 0.02 | 0.00 | 3.02 | 0.10 |
| 67 | 0.61 | 85.13 | 0.00 | 1.56 | 1.03 | 0.46 | 0.94 | 0.05 | 0.12 | 0.02 | 0.02 | 0.00 | 3.02 | 0.10 |
| 68 | 0.38 | 91.85 | 0.00 | 4.85 | 1.83 | 0.25 | 5.40 | 0.34 | 0.19 | 0.06 | 0.02 | 0.00 | 2.53 | 0.15 |
| 68 | 0.38 | 91.85 | 0.00 | 4.85 | 1.83 | 0.25 | 5.40 | 0.34 | 0.19 | 0.06 | 0.02 | 0.00 | 2.53 | 0.15 |
| 69 | 0.50 | 80.01 | 0.00 | 1.05 | 2.52 | 0.56 | 1.51 | 0.07 | 0.19 | 0.03 | 0.02 | 0.00 | 2.23 | 0.07 |
| 69 | 0.50 | 80.01 | 0.00 | 1.05 | 2.52 | 0.56 | 1.51 | 0.07 | 0.19 | 0.03 | 0.02 | 0.00 | 2.23 | 0.07 |
| 70 | 0.27 | 76.76 | 0.00 | 2.10 | 0.92 | 0.59 | 3.06 | 0.17 | 0.27 | 0.03 | 0.02 | 0.00 | 2.15 | 0.07 |
| 70 | 0.27 | 76.76 | 0.00 | 2.10 | 0.92 | 0.59 | 3.06 | 0.17 | 0.27 | 0.03 | 0.02 | 0.00 | 2.15 | 0.07 |
| 71 | 0.45 | 80.72 | 0.00 | 2.67 | 1.61 | 0.40 | 1.83 | 0.14 | 0.22 | 0.03 | 0.02 | 0.00 | 2.39 | 0.10 |
| 71 | 0.45 | 80.72 | 0.00 | 2.67 | 1.61 | 0.40 | 1.83 | 0.14 | 0.22 | 0.03 | 0.02 | 0.00 | 2.39 | 0.10 |

As there was insufficient plant material to cover the bottom of a 50 mm rotating cup for many of the samples, tests were only performed using a 20 mm stationary vial for these samples. NIR spectra were collected and trimmed, retaining the 9040-3275 cm¹ spectral region, and analysed as previously described.

### A. Type Prediction

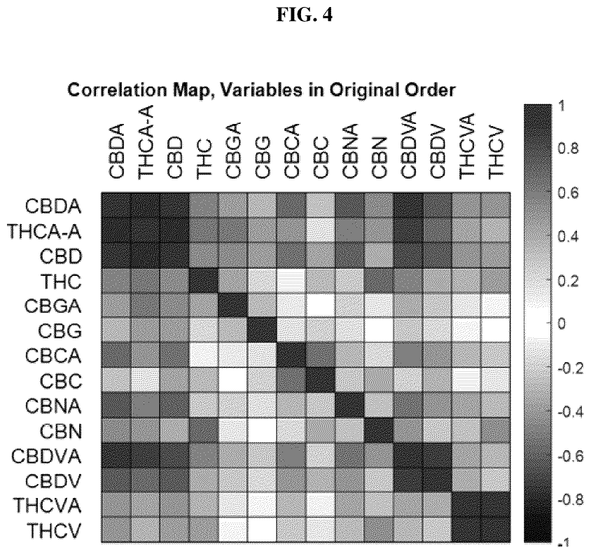

The larger number of samples available meant that the data could be split into a calibration set and a prediction set. This split was calculated automatically using the onion method (keeping outside covariance samples plus random inner space samples) using 70% of the samples for the calibration set. Using this approach, the strains were accurately predicted to be either Type I or Type II via PLSDA modelling (FIG. 4).

The PLSDA models included spectral pre-processing: detrend, OSC (Orthogonal Signal Correction), 1st Derivative (order: 2, window: 15 pt, tails: polyinterp) and mean center. Error of classification after cross validation (venetian blinds with 10 splits and 1 sample per split) for the calibration set was 0%. The samples that had not been used in creating the model were also predicted with 100% accuracy (specificity and sensitivity were both 1). Permutation testing (50 iterations) confirmed that the model was not overfitted (p<0.05).

### B. Cannabinoid Concentration

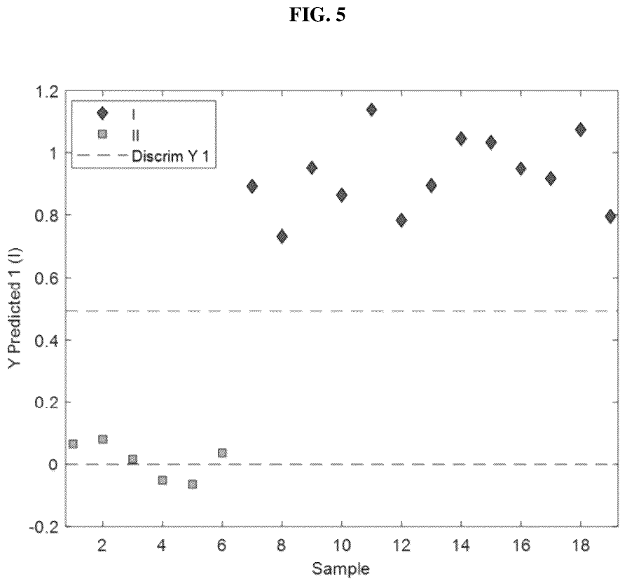

For initial model developed the entire data set was used with pre-processing including detrend, SNV, 2nd Derivative (order: 2, window: 5 pt, tails: polyinterp), Mean Center and cross validation using venetian blinds with 10 splits and 1 sample per split. This method gave good predictions for the major cannabinoids THCA-A and CBDA (FIG. 5).

Using the same parameters, the minor cannabinoids (CBGA, CBCA, CBNA, CBD, and CBDV) were less well predicted, with R² between 0.5 and 0.89, whereas the analysis of THCVA, THC, CBG, CBC CBN and THCV provided R² between 0.23 and 0.49. These predictions may be improved by individually optimising the math treatment for spectral pre-processing. For example, the prediction for CBGA of R²=0.52 may be increased to 0.74 by detrend, EMSC (Extended Scatter Correction), Mean Center, Smoothing (order: 1, window: 15 pt, tails: polyinterp), 1st Derivative (order: 3, window: 15 pt, tails: weighted). A larger data set would also be useful to improve these correlations.

### Discussion

These data show that NIR spectroscopy allows the classification of cannabis by Type I, Type II or Type III and for prediction of cannabinoid content (including cannabinoid in acid form) in cannabis plant material. These studies also demonstrate, for the first time, that a fibre optic probe can be employed to provide sufficient data to allow the classification of cannabis by Type I, Type II or Type III and for prediction of cannabinoid content, as compared to the use of rotating cups. Extrapolating from this, portable, hand held spectrometers can be used to carry out real time monitoring of cannabis plant material for type identification and cannabinoid content. This has applications in large scale breeding, in field and in glasshouse/greenhouse monitoring for optimal harvesting and as a rapid testing tool for authorities.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A method for determining a chemotypic profile of cannabis plant material, the method comprising:
(a) providing a predetermined association between spectroscopic data from reference cannabis plant material and a chemotypic profile from the reference cannabis plant material, wherein the chemotypic profile evaluates cannabinoids THCA-A, CBDA, CBGA, CBCA and CBNA, and wherein the spectroscopic data is near infrared (NIR) spectroscopic data;
(b) providing a sample of cannabis plant material and measuring NIR spectrum to obtain spectroscopic data from at least one region of sample cannabis plant material; and
(c) comparing the NIR spectroscopic data obtained in step (b) with the spectroscopic data of the predetermined association in step (a) to determine the chemotypic profile of the sample cannabis plant material;
wherein the NIR spectrum is measured using a hand-held device; wherein the reference or sample cannabis plant material is derived from a female plant; wherein the reference or sample cannabis plant material is an inflorescence or a leaf; and
wherein the sample cannabis plant material is provided without sample preparation.

2. The method of claim 1, wherein the spectroscopic data is measured by Fourier-transform near infrared (FT-NIR) spectroscopy.

3. The method of claim 1, wherein the NIR spectroscopic data is measured using:
(i) a rotary cup; or
(ii) a fibre optic probe.

4. The method of claim 2, wherein the NIR spectroscopic data measured using the hand-held device is processed in a control unit, wherein the control unit is configured to receive and process the measured spectroscopic data to determine the chemotypic profile of the sample cannabis plant material based on the predetermined association between spectroscopic data from reference cannabis plant material and a chemotypic profile from the reference cannabis plant material.

5. The method of claim 1, wherein the reference or sample cannabis plant material is an inflorescence.

6. The method of claim 1, wherein the spectroscopic data is obtained from reference or sample cannabis plant material that has not been heat treated.

7. The method of claim 1, wherein the chemotypic profile further evaluates at least one cannabinoid in neutral form.

8. The method of claim 7, wherein the at least one cannabinoid in neutral form is selected from the group consisting of CBD, THC, CBG and THCV.

9. The method of claim 8, wherein the at least one cannabinoid in neutral form is selected from the group consisting of CBD and CBDV.

10. The method of claim 1, further comprising classifying the sample cannabis plant material into Type I, Type II or Type III cannabis plant material based on the chemotypic profile of the sample cannabis plant material.

11. The method of claim 1, wherein the predetermined association is a trained classifier.

12. The method of claim 11, wherein the classifier is trained using NIR spectroscopic data from a plurality of reference cannabis plant material and chemotypic profiles from the plurality of reference cannabis plant material.

13. The method of claim 12, wherein the classifier is trained using Partial Least Squares Discriminant Analysis (PLS-DA) with or without venetian blinds cross validation.

14. The method of claim 1, wherein the NIR spectroscopic data obtained in step (b) is pre-processed prior step (c).

15. The method of claim 1, further comprising:
(d) determining a chemotypic profile of a further sample of cannabis plant material derived from the same cannabis plant of the sample of cannabis plant material of step (b) at a subsequent time point in the growth cycle of the cannabis plant;
(e) comparing the chemotypic profiles determined at (c) and (d) to evaluate whether there has been a change to the chemotypic profile of the cannabis plant.

16. A method of selecting growing conditions that favour the development of a cannabis plant with a desirable chemotypic profile, the method comprising:
(a) exposing a first cannabis plant to a first set of selected growing conditions for a period of time;
(b) exposing a second cannabis plant to a second set of selected growing conditions for a period of time, wherein the second set of selected growing conditions is different from the first set of selected growing conditions;
(c) optionally, repeating step (b) for a subsequent set of growing conditions that is different from the first and second sets of selected growing conditions;
(d) determining chemotypic profiles of plant material derived from each of the cannabis plants exposed to the set of selected growing conditions of steps (a)-(c) in accordance with the method of claim 1; and
(e) selecting from the set of growing conditions of steps (a)-(c) one or more sets of selected growing conditions that favour the development of a cannabis plant with a desirable chemotypic profile based on the chemotypic profiles determined at step (d).

17. A method of training a classifier to determine a chemotypic profile of sample cannabis plant material, the method comprising:
(a) obtaining spectroscopic data from cannabis plant material derived from a plurality of cannabis plants and chemotypic profiles from the cannabis plant material, wherein the chemotypic profiles evaluate at least one cannabinoid in acid form;
(b) for each of the plurality of cannabis plants, using a processor, generating an association between the spectroscopic data and the chemotypic profile;
(c) using the association generated in step (b) to train the classifier to determine the chemotypic profile of a sample cannabis plant material from spectroscopic data, optionally wherein the classifier is trained using a Partial Least Squares Discriminant Analysis (PLS-DA); optionally further comprising venetian blinds cross validation; and
(d) optionally, repeating steps (a)-(c) using a different plurality of cannabis plants to improve the accuracy of the classifier.

18. The method of claim 17, further comprising:
(e) utilising a classifier trained in accordance with steps (a)-(c) or steps (a)-(d) to determine the chemotypic profile of the cannabis plant material from the spectroscopic data, wherein the chemotypic profile evaluates at least one cannabinoid in acid form, preferably wherein the chemotypic profile evaluates the concentration of the cannabinoid in the plant material;
(f) outputting the chemotypic profile; and
(g) optionally, classifying the plant material into Type I, Type II or Type III cannabis plant material based on the chemotypic profile of the plant material.

19. A method of claim 2, wherein the spectroscopic data is measured with a resolution of 8 cm⁻¹.

20. A method of claim 5, wherein the reference or sample cannabis plant material comprises cannabis trichomes.

21. The method of claim 14, wherein one or more or all of the following apply:
(i) the pre-processing limits the spectroscopic data obtained in step (b) to a spectrum of from about 3500 cm⁻¹ to about 12,500 cm⁻¹;
(ii) the pre-processing limits the spectroscopic data obtained in step (b) to a spectrum of from about 3500 cm⁻¹ to about 9250 cm⁻¹; and
(iii) the pre-processing comprises one or more methods selected from the group consisting of: detrend, extended scatter correction (EMSC), orthogonal signal correction (OSC), 1st or 2nd derivative, smoothing, and mean center.
